(19) United States
(12) Patent Application Publication
(10) Pub. No.: US 2007/0148193 A1
(43) Pub. Date: Jun. 28, 2007

(54) Lutein concentrate

(75) Inventor: Dariush Behnam, Rossdorf (DE)

Correspondence Address: JACOBSON HOLMAN PLLC, 400 SEVENTH STREET N.W., SUITE 600, WASHINGTON, DC, 20004, US

(73) Assignee: AQUANOVA GERMAN SOLUBILISATE TECHNOLOGIES (AGT) GM, Darmstadt (DE)

(21) Appl. No.: 10/576,272

(22) PCT Filed: Nov. 18, 2004
(86) PCT No.: PCT/EP04/13105

§ 371 Date: Apr. 17, 2006

(30) Foreign Application Priority Data
Dec. 10, 2003 (DE) 203 19 249.4

(51) Int. Cl. A61K 31/015; A61K 9/00

(52) U.S. Cl. 424/400; 514/763

(57) ABSTRACT

The invention relates to a lutein solubilized product comprised of one part, which consists of a lutein-containing composition, and of approximately 7 to approximately 9 parts polysorbate, and to a method for the production thereof, during which a lutein-containing composition is mixed with approximately seven to nine times the amount of polysorbate until obtaining homogeneity.

The present invention relates to a lutein concentrate and a method for its manufacture.

The xanthophyll lutein is a natural coloring agent which is also permissible and usable in food. It occurs in many plants and algae (such as Dunaliella salina) and may be obtained therefrom in its esterified form, for example, as a palmitate or myristate, in the way described in U.S. Pat. No. 6,191,293. The lutein ester is often mixed with a small component of zeaxanthin ester.

Since lutein is recognized to physiologically counteract macular degeneration and also plays a role in atherogenesis, lutein preparations are often recommended as a component of nutritional supplements. The use of compositions containing lutein is made more difficult because lutein and its ester are practically insoluble in water and therefore may only be processed in oleaginous suspension. Therefore, the lutein component actually usable by the organism is very small.

The present invention is based on the object of improving the bioavailability of compositions containing lutein or its ester and their processability.

A lutein solubilizate, which comprises one part of a composition containing lutein and approximately 7 to approximately 9 parts of a polysorbate, expediently polysorbate 80, is used for this purpose according to the present invention. In particular, a solubilizate which comprises approximately 3 weight-percent to approximately 15 weight-percent of the composition and approximately 75 weight-percent to approximately 85 weight-percent of the polysorbate has proven itself. Further advantageous embodiments are specified in the subclaims.

The solubilizate, which contains approximately 1 weight-percent to approximately 3 weight-percent pure lutein, has a dark red color and is soluble without anything further in water at room or body temperature or possibly at a slightly elevated temperature. The water solubility of the solubilizate not only makes it easier to process as a food coloring agent and as a component of nutritional supplements, but rather also increases its bioavailability.

A study by J. Milde and E. F. Elstner (Technical University of Munich) has shown that lutein has no effect on the oxidation resistance of LDL (low-density lipoprotein) as a powder or dissolved in ethanol. In contrast, the lutein solubilizate according to Example 3 indicated below, for example, results in a significantly increased oxidation protection. The micellated lutein from the solubilizate is enriched in the LDL by a factor of 9 more (10 mole lutein/mole LDL). A further study (Biesalski, Technical University of Stuttgart) displays a sixfold better bioavailability of the lutein solubilizate according to the present invention.

In the aqueous solution of the lutein concentrate, the lutein in micellated form is provided having a micelle size of approximately 50 nm to approximately 100 nm.

If the lutein solubilizate according to the present invention also contains approximately 10.0 weight-percent of a medium-chain triglyceride, the manufacturing of the concentrate is simplified. The polysorbate is especially expediently polysorbate 80. Favorable solubilizates having equally good properties as with the use of polysorbate 80 may also be obtained using polysorbate 20.

The method according to the present invention for manufacturing the solubilizate provides that a composition containing lutein is stirred with approximately seven to approximately nine times the quantity of polysorbate, possibly with slight heating.

The solubilizate according to the present invention may be processed especially easily by heating it to at least 45° C., expediently approximately 70 to approximately 80° C., and stirring it with correspondingly heated water. Two parts water are used for one part solubilizate in this case.

Preferred embodiments of the present invention are also specified in the subclaims. Three manufacturing examples are described in the following:

### EXAMPLE 1

Approximately 100 g water is heated to approximately 50° C. and approximately 50 g of a powdered composition which contains approximately 20% lutein and 17% zeaxanthin (reference source: Pfannenschmidt GmbH, Hamburg, product name: LUTEIN 20% extract powder), is added to the heated water. The mixture is stirred strongly for approximately five minutes while maintaining the temperature. Subsequently, approximately 850 g polysorbate 80 is heated to approximately 100° C. and added to the mixture. The resulting overall mixture is stirred at approximately 100° C. until a homogeneous, deep red-brown, water-soluble product results. After cooling to room or body temperature, the water solubility of the aqueous phase obtained, having a 1.0 weight-percent lutein content, is maintained.

### EXAMPLE 2

32.5 g of a powdered composition (reference source: Cognis, Xangold Grade Lec.) having a lutein ester content of approximately 60% and a lutein equivalent of approximately 35% and a total zeaxanthin content of at most approximately 7% is mixed with 117.5 g water and the mixture is heated to 45° C. 750 g polysorbate 80 is mixed with 100 g of a moderate-length-chain triglyceride fat (reference source: CAESAR & LORETZ GmbH, Hilden, product: MIGLYOL 812) and heated to approximately 60° C. Both mixtures are poured together and heated to 90° C. and stirred continuously at the same time. After cooling to room or body temperature, a dark red, solid, water-soluble solubilizate results. Heating of the solubilizate to approximately 45° C. makes it liquid. The solubilizate described above has a total lutein equivalent concentration of approximately 1%.

### EXAMPLE 3

150.0 g of a composition comprising a mixture of vegetable oil (e.g., sunflower seed oil) and approximately 20% lutein (reference source: Hoffman-LaRoche, Basel) is heated to approximately 85° C. and subsequently added to 850 g polysorbate 80 which has previously been heated to 85° C. The overall mixture is stirred while maintaining the temperature until a homogeneous, deep red-brown, water-soluble solubilizate results. After cooling to room or body temperature, the water solubility of the aqueous phase, having a 3 weight-percent lutein content, thus obtained is maintained.

The same lutein concentrations are achieved in the solubilizates if polysorbate 80 is replaced by polysorbate 20. Solubilizates manufactured according to Example 3 may preferably be diluted with temperature-controlled water, for the purpose of easier processing and/or final processing into the final product, until a 1% lutein solubilizate is thus obtained. For this purpose, the solubilizate, which previously had been heated to approximately 45° C., is mixed in a ratio of 1:2 with water at the same temperature. It is to be ensured in this case that the 3% solubilizate is added to the water and stirred and not the reverse.

The numbers specified above are those which result in a solubilizate having optimum properties. However, the present invention is not restricted to these precise numerical values. Deviations of approximately 10% in each case still result in solubilizates having satisfactory properties as defined by the object of the present invention.

1. A lutein solubilizate, comprising one part of a composition containing lutein and approximately 7 to approximately 9 parts of a polysorbate.

2. The solubilizate according to claim 1, comprising approximately 3 weight-percent to approximately 15 weight-percent of the composition and approximately 75 weight-percent to approximately 85 weight-percent of the polysorbate.

3. The solubilizate according to claim 1, containing polysorbate 80 as the polysorbate.

4. The solubilizate according to claim 1, also comprising approximately 10 weight-percent moderate-length-chain triglyceride.

5. The solubilizate according to claim 1, wherein the composition is obtained from plants and/or algae.

6. The solubilizate according to claim 1, which contains approximately 10 weight-percent to approximately 15 weight-percent water.

7. The solubilizate according to claim 1, wherein the composition contains approximately 10 weight-percent to approximately 30 weight-percent lutein.

8. The solubilizate according to claim 1, wherein the composition contains up to approximately 1 weight-percent zeaxanthin.

9. A method for manufacturing a lutein solubilizate according to claim 1, wherein a composition containing lutein is stirred with approximately seven to nine times the quantity of polysorbate until homogeneity is reached.

10. The method according to claim 9, wherein approximately 5 weight-percent to approximately 15 weight-percent of the composition is stirred with approximately 75 to approximately 85 weight-percent of the polysorbate.

11. The method according to claim 9, wherein the stirring is performed at elevated temperature of approximately 90° C. to approximately 100° C.

12. The method according to claim 9, wherein the powdered composition is stirred with water at a temperature of approximately 45° C. to approximately 50° C.

13. The method according to claim 12, wherein the composition is stirred into approximately 10 weight-percent to approximately 15 weight-percent water.

14. The method according to claim 9, wherein the polysorbate is mixed with a moderate-length-chain triglyceride at a temperature of approximately 60° C. before it is mixed with the composition.
